(19) United States
(12) Patent Application Publication
(10) Pub. No.: US 2020/0375475 A1
(43) Pub. Date: Dec. 3, 2020

(54) Biological Information Measurement Device

(71) Applicant: Seiko Epson Corporation, Tokyo (JP)

(72) Inventor: Katsutoshi MATSUURA, Chino (JP)

(21) Appl. No.: 16/886,846

(22) Filed: May 29, 2020

(30) Foreign Application Priority Data
May 30, 2019 (JP) 2019-101035

(51) Int. Cl. A61B 5/026; A61B 5/00; A61B 5/01; A61B 5/145

(52) U.S. Cl. CPC A61B 5/0261; A61B 5/6824; A61B 2562/16; A61B 5/145; A61B 2562/0233; A61B 5/01

(57) ABSTRACT

A biological information measurement device includes a light emitting section configured to emit irradiation light with which an arm is irradiated, a light receiving section configured to receive the reflected light which is the irradiation light reflected by the arm, a passage part which the irradiation light and the reflected light pass through, The light blocking section configured to block the irradiation light propagating from the light emitting section toward the light receiving section, and a back lid which is opaque, and supports the passage part, wherein in a plan view viewed from a first direction from the light emitting section toward the passage part, the light blocking section is a metal plate disposed between the light emitting section and the light receiving section, and in a first side surface through a fourth side surface of the light receiving section facing to directions crossing the first direction, the second side surface through the fourth side surface 6h not opposed to the light blocking section are opposed to the back lid.

The present application is based on, and claims priority from, JP Application Serial Number 2019-101035, filed May 30, 2019, the disclosure of which is hereby incorporated by reference herein in its entirety.

### BACKGROUND

### 1. Technical Field

The present disclosure relates to a biological information measurement device.

### 2. Related Art

In the past, there has been known a biological information measurement device for measuring the pulse as a type of biological information. International Publication No. WO 2017/094089 (Document 1) discloses a photosensor used in the biological information measurement device. According to Document 1, the photosensor is provided with a light emitting element as a light emitting section and a light receiving element as a light receiving section. The light emitting element is provided with an LED (Light Emitting Diode). The light emitting element emits irradiation light to a living body through light transmissive resin. The light receiving element converts reflected light entering the light receiving element through the light transmissive resin into a pulse wave signal as an electric signal.

The living body includes a blood vessel through which the blood flows. The pulsation of the blood vessel coordinates with the cardiac motion. Since a part of the light emitted by the light emitting section is absorbed by the blood, the light receiving section receives the reflected light reflecting the pulsation of the blood vessel. In other words, the intensity of the reflected light received by the light receiving section reflects the pulsation of the blood vessel. Further, the pulse wave signal is made to be a signal reflecting the pulsation of the blood vessel.

The living body is irradiated with the irradiation light which is emitted by the light emitting element and passes through the light transmissive resin. The light receiving element is irradiated with a part of the reflected light which is reflected by the living body and passes through the light transmissive resin. The light receiving element receives the reflected light with which the light receiving element is irradiated. The irradiation light emitted by the light emitting element spreads as the irradiation light proceeds. Therefore, the shorter the distance between the light emitting element and the living body is, the higher the intensity of the irradiation light with which the living body is irradiated becomes. Further, the reflected light reflected by the living body also spreads as the reflected light proceeds. Therefore, the shorter the distance between the living body and the light receiving element is, the higher the intensity of the reflected light received by the light receiving element becomes.

The shorter the distance between the light emitting element and the living body is, the higher the intensity of the reflected light received by the light receiving element can be made. Further, the higher the intensity of the reflected light received by the light receiving element is, the higher the ratio of the pulse wave signal to the noise can be made.

In the photosensor described in Document 1, a wall made of light blocking resin is disposed between the light emitting element and the light receiving element. The wall made of light blocking resin blocks the irradiation light emitted by the light emitting element to prevent the light receiving element from being directly irradiated with the irradiation light. Since the light blocking resin made too thin lacks the enough light blocking property, it is necessary to thicken the thickness of the wall. Since the light blocking resin is thick, the distance between the light emitting element and the light receiving element has become long. When the distance between the light emitting element and the light receiving element is long, the total distance of the propagation distance of the irradiation light and the propagation distance of the reflected light is elongated compared to when the distance between the light emitting element and the light receiving element is short. When the propagation distance of the light is elongated, the intensity of the reflected light to be received by the light receiving element decreases. When the intensity of the reflected light to be received by the light receiving element is low, the accuracy of detecting the pulse degrades. As described above, since the distance between the light emitting element and the light receiving element is long, there is a limitation in improving the accuracy of detecting the pulse.

### SUMMARY

A biological information measurement device according to the present disclosure includes a light emitting section configured to emit irradiation light with which a living body is irradiated, a light receiving section configured to receive reflected light which is the irradiation light reflected by the living body, a passage part which the irradiation light and the reflected light pass through, a light blocking section configured to block the irradiation light propagating from the light emitting section toward the light receiving section, and a back lid which is opaque, and supports the passage part, wherein the light blocking section is a metal plate disposed between the light emitting section and the light receiving section in a plan view viewed from a first direction from the light emitting section toward the passage part, and a side surface which is one of side surfaces of the light receiving section, which faces to a direction crossing the first direction, and which fails to be opposed to the light blocking section, is opposed to the back lid.

In the biological information measurement device described above, in a plan view viewed from the first direction, the side surface of the light receiving section and the back lid may be separated from each other.

In the biological information measurement device described above, in a plan view viewed from the first direction, the side surface of the light receiving section and the back lid may have contact with each other.

In the biological information measurement device described above, in a plan view viewed from the first direction, the light receiving section and a part of the back lid may overlap each other on the living body side of the light receiving section.

In the biological information measurement device described above, the passage part may be recessed in an inner surface facing to the light blocking section, and a side of the light blocking section facing to the passage part may protrude along the inner surface.

### BRIEF DESCRIPTION OF THE DRAWINGS

FIG. 1 is a schematic perspective view showing a configuration of a biological information measurement device according to a first embodiment.

FIG. 2 is a schematic perspective view for explaining a mounting state of the biological information measurement device.

FIG. 3 is a schematic plan view showing a structure of the biological information measurement device.

FIG. 4 is a schematic sectional side view showing the structure of the biological information measurement device.

FIG. 5 is a schematic perspective view showing a structure of a sensor part.

FIG. 6 is a schematic sectional side view of a principal part for explaining propagation paths of the light.

FIG. 7 is a schematic sectional side view of a principal part for explaining propagation paths of the light.

FIG. 8 is a schematic sectional side view showing a structure of a light receiving section.

FIG. 9 is a schematic view for explaining a method of detecting the pulsation of a blood vessel.

FIG. 10 is a diagram for explaining a relationship between a blood vessel transmural pressure difference and an intravascular volume.

FIG. 11 is a diagram showing a temporal change in the intravascular volume.

FIG. 12 is a block diagram of electric control of the biological information measurement device.

FIG. 13 is a schematic sectional side view showing a principal part of a configuration of a sensor part and a back lid related to a second embodiment.

FIG. 14 is a schematic sectional side view showing a principal part of the configuration of the sensor part and the back lid.

FIG. 15 is a schematic sectional side view showing a principal part of a configuration of a sensor part and a back lid related to a third embodiment.

FIG. 16 is a schematic sectional side view showing a principal part of the configuration of the sensor part and the back lid.

FIG. 17 is a schematic view for explaining a mounting state of a biological information measurement device according to a fourth embodiment.

FIG. 18 is a schematic perspective view showing a configuration of the biological information measurement device.

### DESCRIPTION OF EXEMPLARY EMBODIMENTS

Hereinafter, some embodiments will be described along the accompanying drawings. It should be noted that the members in each of the drawings are illustrated with different scales from each other in order to provide a size large enough to be recognized in the drawing.

### First Embodiment

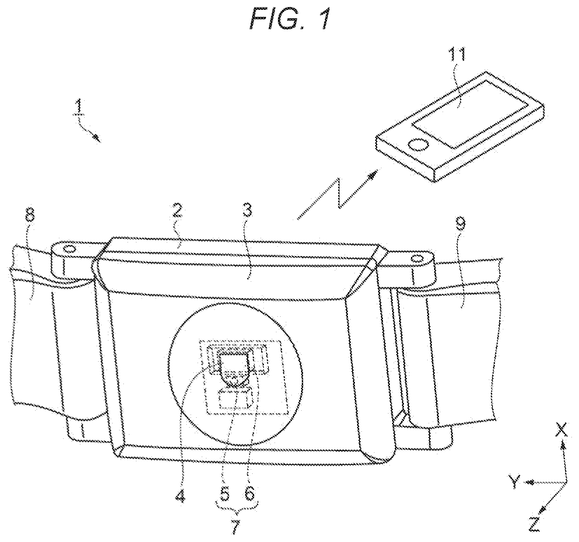

In the present embodiment, a characteristic example of a biological information measurement device for detecting the pulsation of a blood vessel will be described along FIG. 1 through FIG. 12. FIG. 1 is a schematic perspective view showing a configuration of the biological information measurement device. As shown in FIG. 1, the biological information measurement device 1 is provided with a case 2 shaped like a box having a predetermined thickness. On one side in the thickness direction of the case 2, there is disposed a back lid 3. The back lid 3 is provided with a passage part 4 through which light can pass. Inside the case 2, there are disposed a sensor part 7 provided with a light emitting section 5 and a light receiving section 6 and so on. The light emitting section 5 emits irradiation light with which a living body is irradiated. Reflected light which is the irradiation light reflected inside the living body is received by the light receiving section 6.

On side surfaces of the case 2, there are disposed a first band 8 and a second band 9 so as to be located on both sides of the case 2. At one end of the first band 8, there is disposed a coupling section not shown for coupling the first band 8 and the second band 9 to each other. In the drawing, a direction from the light emitting section 5 toward the light receiving section 6 is defined as an X direction. A direction from the second band 9 toward the first band 8 is defined as a Y direction. A direction from the case 2 toward the back lid 3 is defined as a Z direction. The X direction, the Y direction, and the Z direction are arranged to be directions perpendicular to each other. A direction opposite to the X direction is defined as a −X direction. A direction opposite to the Y direction is defined as a −Y direction. A direction opposite to the Z direction is defined as a −Z direction.

The biological information measurement device 1 is provided with a function of performing wireless communication. Further, the biological information measurement device 1 transmits the pulse data measured to electronic equipment such as a smartphone 11 with the wireless communication. Further, the smartphone 11 displays the pulse data measured by the biological information measurement device 1.

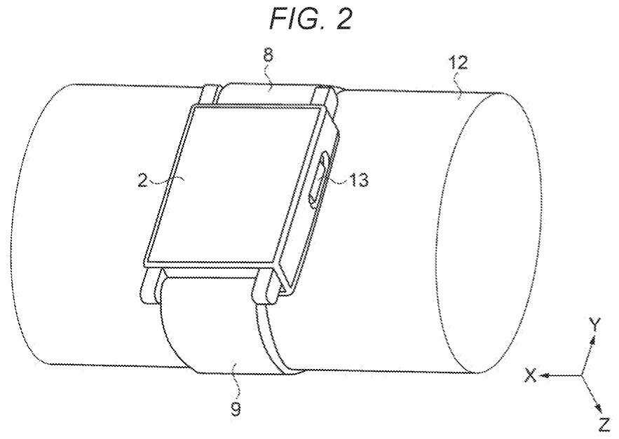

FIG. 2 is a schematic perspective view for explaining a mounting state of the biological information measurement device. As shown in FIG. 2, the biological information measurement device 1 is mounted on an arm 12 as the living body of a human body. The first band 8 and the second band 9 are wound around the arm 12, and then the first band 8 and the second band 9 are coupled to each other with the coupling section. As described above, the biological information measurement device 1 is wearable equipment which is mounted on the arm 12 to measure the biological information of the human body. The biological information measurement device 1 detects a pulse wave signal to calculate a pulse rate. It should be noted that the pulse wave signal is obtained by observing a pressure change or a volumetric change in the pulsation of the blood vessel. The pulse rate is the number of peaks of the pulse wave signal included in one minute.

The biological information measurement device 1 is mounted so that the back lid 3 has contact with the arm 12. On this occasion, the back lid 3 and the passage part 4 have contact with the arm 12. On the side surface of the case 2, there is disposed an external connector 13 compliant with the USB (Universal Serial Bus). The biological information measurement device 1 is charged through the external connector 13.

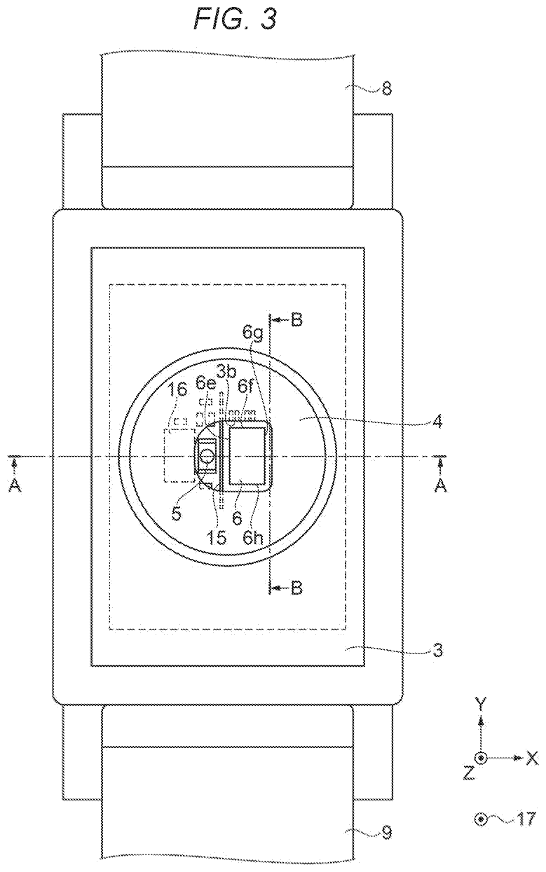

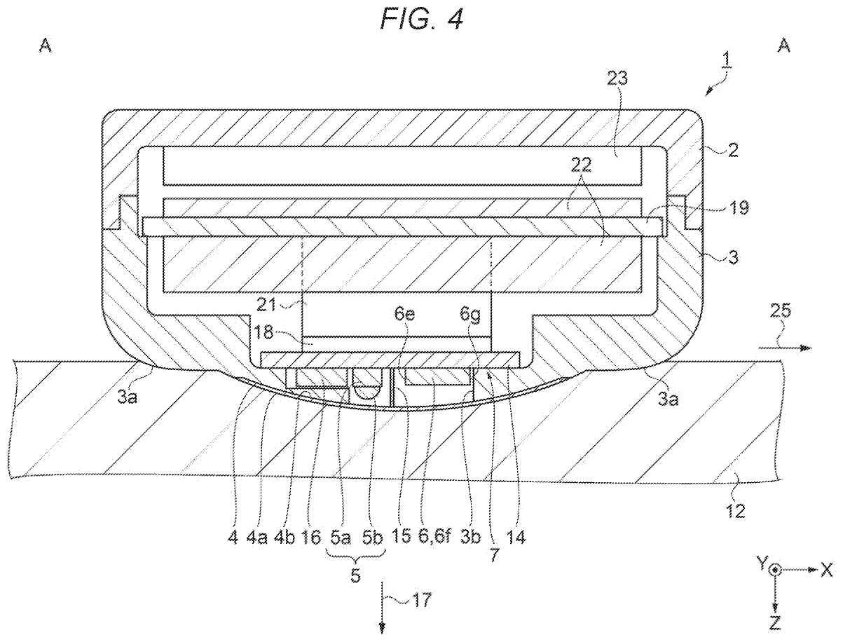

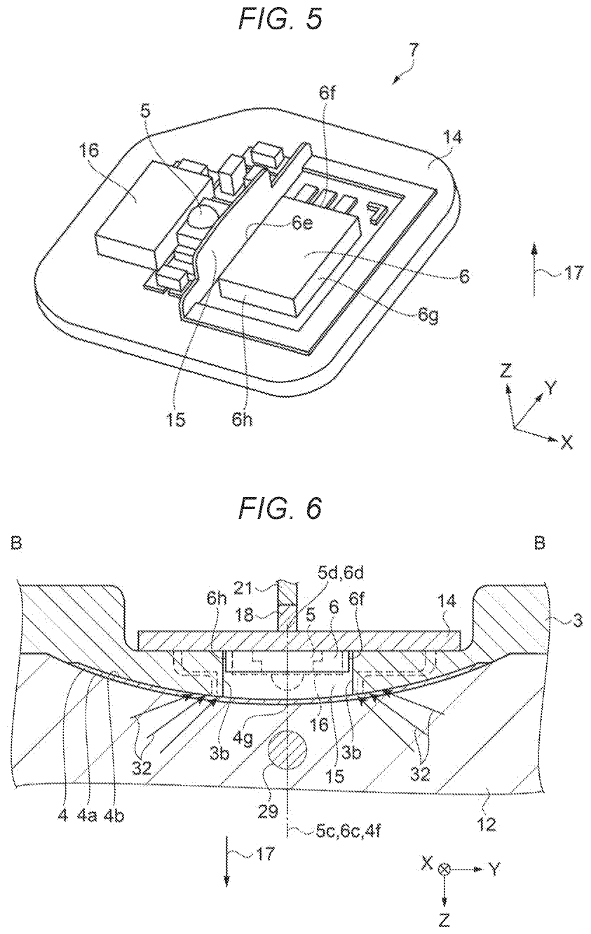

FIG. 3 is a schematic plan view showing a structure of the biological information measurement device, and is a diagram of the biological information device 1 viewed from the back lid 3 side. FIG. 4 is a schematic sectional side view showing a structure of the biological information measurement device, and is a diagram viewed from a cross-sectional surface along the line A-A shown in FIG. 3. FIG. 5 is a schematic perspective view showing a structure of a sensor part.

As shown in FIG. 3 through FIG. 5, the external shape of the passage part 4 is a circular shape. A surface on the Z direction side of the passage part 4 protrudes toward the Z direction. A surface on the −Z direction side of the passage part 4 is recessed toward the Z direction. Therefore, the passage part 4 has a plate-like shape. The back lid 3 supports the passage part 4 from the −Z direction side. The back lid 3 is opaque. The back lid 3 is provided with a hole 3b disposed on the Z direction side of the light emitting section 5 and the light receiving section 6. Since the passage part 4 is overlaid on the back lid 3 so as to cover the hole 3b, the hole 3b is blocked by the passage part 4. The passage part 4 is transparent, and therefore, the light emitting section 5, the light receiving section 6, and a light blocking section 15 can be seen through the hole 3b. Therefore, in FIG. 3, the light emitting section 5, the light receiving section 6, and the light blocking section 15 are represented by the solid lines.

The sensor part 7 is disposed on the passage part 4 side of the inside surrounded by the case 2, the back lid 3, and the passage part 4. The sensor part 7 is provided with a sensor board 14 supported by the back lid 3. The sensor board 14 is a rigid board. On the passage part 4 side of the sensor board 14, there are disposed the light emitting section 5, the light receiving section 6, the light blocking section 15, and a drive section 16.

The light emitting section 5 emits the irradiation light with which the arm 12 is irradiated. The light emitting section 5 is constituted by a light emitting body 5a, a lens body 5b, and so on. The light emitting body 5a is an LED (Light Emitting Diode) chip having a light emitting element such as an LED encapsulated with resin. The light emitting body 5a can also be a bare chip of the light emitting element not encapsulated with encapsulation resin. The light emitted by the light emitting body 5a is green light. Since the green light is reflected by a shallow part of the skin, it is possible to irradiate an arteriole with the green light. It should be noted that the light emitted by the light emitting body 5a can be other light than the green light.

The lens body 5b converges the irradiation light on a predetermined depth in the arm 12. The predetermined depth means the depth where the arterioles exist. The material of the lens body 5b is not particularly limited providing the material has a light transmissive property, and for example, acrylic resin, epoxy resin, and glass can be used as the material.

The light receiving section 6 receives the reflected light which is the irradiation light reflected by the arm 12. Further, the light receiving section 6 outputs a detection signal representing an amount of the reflected light received. The detection signal corresponds to the pulse wave signal. The light receiving section 6 is a PD (Photodiode) chip having a light receiving element as a PD encapsulated with encapsulation resin although the detailed illustration will be omitted. The light receiving section 6 can also be a bare chip of the light receiving element not encapsulated with the encapsulation resin.

In a plan view from a first direction 17, the light receiving section 6 is a rectangular solid having a plane shaped like a rectangular plate. A side surface of the light receiving section 6 facing to the −X direction is defined as a first side surface 6e. The first side surface 6e is opposed to the light blocking section 15. A side surface of the light receiving section 6 facing to the Y direction is defined as a second side surface 6f. A side surface of the light receiving section 6 facing to the X direction is defined as a third side surface 6g. A side surface of the light receiving section 6 facing to the −Y direction is defined as a fourth side surface 6h. The first side surface 6e, the second side surface 6f, the third side surface 6g, and the fourth side surface 6h correspond to side surfaces of the light receiving section 6 facing to directions crossing the first direction 17. The second side surface 6f, the third side surface 6g, and the fourth side surface 6h not opposed to the light blocking section 15 are each opposed to a side surface of the hole 3b of the back lid 3.

The light receiving element has an n-type semiconductor region on a silicon substrate side, and a p-type semiconductor region on a light receiving surface side. When the light having sufficiently high energy enters the p-type semiconductor region, an electrical current is output due to a photovoltaic effect. The light receiving section 6 is provided with a wavelength filter for passing the light the same in wavelength as the reflected light and preventing the light other than the reflected light from passing therethrough.

The light blocking section 15 is disposed between the light emitting section 5 and the light receiving section 6. A direction from the light emitting section 5 toward the passage part 4 is defined as the first direction 17. The first direction 17 is the same direction as the Z direction. In the plan view from the first direction 17, the light blocking section 15 is a metal plate disposed between the light emitting section 5 and the light receiving section 6. The material of the light blocking section 15 is not particularly limited, but in the present embodiment, nickel silver, for example, is used. The light blocking section 15 is formed using a press machine. A surface of the light blocking section 15 is tin-plated, and is therefore made easy to bond to the sensor substrate 14 with solder. The light blocking section 15 blocks the irradiation light propagating from the light emitting section 5 toward the light receiving section 6. The light blocking section 15 prevents the irradiation light emitted from the light emitting section 5 from directly entering the light receiving section 6 without passing through the arm 12. In addition, the light blocking section 15 prevents stray light other than the reflected light reflected by the arm 12 from entering the light receiving section 6. It is possible to apply a surface treatment for preventing reflection of the light to the light blocking section 15.

The drive section 16 is a circuit for driving the light emitting section 5 and the light receiving section 6. The drive section 16 controls the electric power to be supplied to the light emitting section 5. Further, the drive section 16 controls start and stop of the supply of the electric power. Further, the drive section 16 functions as an AFE (Analog Front End). The drive section 16 amplifies the electric signal output by the light receiving section 6. Then, the drive section 16 is provided with a filter which removes noises included in the electric signal thus amplified. Further, the drive section 16 is provided with an ADC (Analog Digital Converter), and the ADC converts the analog electric signal into an electric signal of digital data, and then outputs the result.

On the surface located on the case 2 side of the sensor board 14, there is disposed a first connector 18. On the case 2 side of the sensor board 14, there is disposed a main board 19. On the surface located on the sensor board 14 side of the main board 19, there is disposed a second connector 21. The second connector 21 and the first connector 18 are electrically coupled to each other.

On both surfaces of the main board 19, there are mounted electric components 22 such as a CPU, a memory, chip resistors, chip capacitors, an antenna. The detection signal representing the amount of the reflected light received is input to the main board 19 from the sensor board 14. Further, the main board 19 calculates the pulse rate. Further, the main board 19 transmits the data of the pulse rate with the wireless communication.

On the case 2 side of the main board 19, there is disposed a secondary cell 23. The secondary cell 23 charges with the electric power supplied from the external connector 13. Further, the secondary cell 23 supplies the electric power to the sensor board 14 and the main board 19. As the secondary cell 23, there is used a lithium cell.

The passage part 4 has a light transmissive property. Therefore, the irradiation light emitted by the light emitting section 5 passes through the passage part 4. Further, the reflected light reflected by the arm 12 also passes through the passage part 4. A part on the first direction 17 side of the passage part 4 is defined as an outer surface part 4a. The outer surface part 4a has contact with the arm 12. Further, a surface in which the back lid 3 has contact with the arm 12 is defined as a contact surface 3a. The outer surface part 4a is a convex surface protruding toward the first direction 17 from the contact surface 3a.

A part forming an opposite surface of the outer surface part 4a in the passage part 4 is defined as an inner surface part 4b as an internal surface. One of directions perpendicular to the first direction 17 is defined as a second direction 25. The second direction 25 is defined as a direction from the light emitting section 5 toward the light receiving section 6. The second direction 25 is the same direction as the X direction. In a cross-sectional view viewed from the second direction 25, the inner surface part 4b forms a concave surface.

The outer surface part 4a is a spherical surface having a dome-like shape. The cross-sectional surface of the passage part 4 is a circular arc, the inner surface part 4b forms a concentric circular arc shape one size smaller than that of the outer surface part 4a, and the thickness of the passage part 4 is made uniform. Since there is a space on the inner surface part 4b side of the passage part 4, it is possible to dispose the light blocking section 15 to the vicinity of the outer surface part 4a.

The light emitting section 5 and the light receiving section 6 are housed in the hole 3b of the back lid 3. A part of the light emitting section 5 protrudes in the first direction 17 from the contact surface 3a. Since there is the space on the inner surface part 4b side of the passage part 4, it is possible to dispose the light emitting section 5 to the vicinity of the outer surface part 4a. Since the distance between the light emitting section 5 and the arm 12 is short, it is possible for the arm 12 to be irradiated with the irradiation light high in intensity.

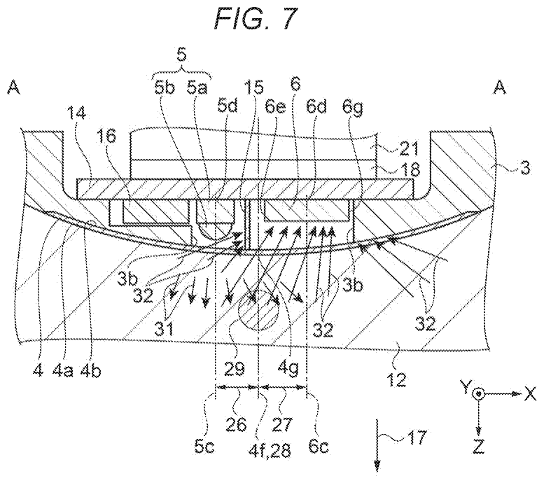

FIG. 6 and FIG. 7 are schematic sectional side views of a principal part for explaining paths of the light. FIG. 6 is a view viewed from a cross-sectional surface side along the line B-B shown in FIG. 3. FIG. 7 is a view viewed from a cross-sectional surface side along the line A-A shown in FIG. 3. As shown in FIG. 6 and FIG. 7, a center line passing through the center 5d of the light emitting section 5 in the plan view from the first direction 17 is defined as a light emitting section center line 5c. The center line 5d of the light emitting section 5 is a centroid of the diagram in the plan view from the first direction 17. In the plan view from the first direction 17, the center line passing through the center 6d of the light receiving section 6 is defined as a light receiving section center line 6c. The center line 6d of the light receiving section 6 is a centroid of the diagram in the plan view from the first direction 17. Further, in the plan view from the first direction 17, a line passing through a crest 4g of the outer surface part 4a and extending in the first direction 17 is defined as a crest indication line 4f. The crest 4g of the outer surface part 4a represents a point protruding the furthest in the first direction 17 of the outer surface part 4a.

A distance between the light emitting section center line 5c and the crest indication line 4f is defined as a first distance 26. A distance between the light receiving section center line 6c and the crest indication line 4f is defined as a second distance 27. In this case, the first distance 26 and the second distance 27 are the same distance.

The crest 4g of the outer surface part 4a applies strong pressure to the arm 12. In the place where the pressure is applied, the change in pulsation of the blood vessel increases. Therefore, the change in pulsation of the blood vessel increases in a part on the crest indication line 4f of the arm 12. A line in the first direction 17 passing midway between the light emitting section center line 5c of the light emitting section 5 and the light receiving section center line 6c of the light receiving section 6c is defined as an intermediate line 28. The intermediate line 28 overlaps the crest indication line 4f. The inside of the arm 12 in the first direction 17 of the intermediate lint 28 and the crest indication line 4f is defined as a measurement target part 29.

The irradiation light 31 emitted by the light emitting section 5 propagates inside the arm 12. Then, a part of the reflected light 32 reflected by the inside of the arm 12 propagates toward the light receiving section 6. A distance obtained by adding a distance from the light emitting section 5 to the measurement target part 29 and a distance from the measurement target part 29 to the light receiving section 6 is defined as a first distance. An arbitrary part other than the measurement target part 29 in the plan view viewed from the first distance 17 is defined as a reference part. Further, a distance obtained by adding a distance from the light emitting section 5 to the reference part and a distance from the reference part to the light receiving section 6 is defined as a second distance. In this case, the first distance is shorter than the second distance. The shorter the distance the light propagates between the light emitting section 5 to the light receiving section 6 is, the higher the intensity of the light received by the light receiving section 6 is.

Therefore, the measurement target part 29 is the place where the biological information measurement device 1 can measure the change in pulsation of the blood vessel with high sensitivity. Since the crest 4g of the outer surface part 4a applies the pressure to the measurement target part 29, it is possible for the biological information measurement device 1 to perform the measurement in the place where the pulsation of the blood vessel changes a lot with high sensitivity. Further, when the outer surface part 4a of the biological information measurement device 1 moves along the surface of the arm 12 in an exercise or the like, the sensor part 7 measures the pulsation of the blood vessel in the measurement target part 29 depressed by the outer surface part 4a. In other words, the biological information measurement device 1 performs the measurement in the place where the pulsation of the blood vessel changes a lot with high sensitivity. Therefore, it is possible for the biological information measurement device 1 to stably measure the pulsation of the blood vessel.

Since the change in intensity of the irradiation light does not reflect the pulse, the irradiation light 31 received by the light receiving section 6 turns to a noise component. When the light receiving section 6 does not receive the irradiation light 31, the accuracy of detecting the pulse becomes higher. A part of the irradiation light 31 propagates toward the light receiving section 6 without passing through the arm 12. The light blocking section 15 is disposed between the light emitting section 5 and the light receiving section 6. The light blocking section 15 blocks the irradiation light 31 propagating toward the light receiving section 6. The light blocking section 15 prevents the irradiation light 31 from being received by the light receiving section 6.

The accuracy of detecting the pulse is higher when the intensity of the reflected light 32 to be received by the light receiving section 6 is higher compared to when the intensity of the reflected light is lower. The shorter the distance between the light emitting section 5 and the measurement target part 29 is, the higher the intensity of the irradiation light 31 with which the measurement target part 29 is irradiated becomes. The shorter the distance between the light receiving section 6 and the measurement target part 29 is, the higher the intensity of the reflected light 32 received by the light receiving section 6 becomes.

In a triangle having the light emitting section 5, the measurement target part 29, and the light receiving section 6 as vertexes, the distance between the light emitting section 5 and the measurement target part 29 corresponds to the propagation distance of the irradiation light 31. The distance between the light receiving section 6 and the measurement target part 29 corresponds to the propagation distance of the reflected light 32. When the distance between the light emitting section 5 and the light receiving section 6 is short, the first distance can be shortened compared to when the distance between the light emitting section 5 and the light receiving section 6 is long. Since the irradiation light 31 and the reflected light 32 do not have a converging property, the shorter the first distance is, the higher the intensity of the reflected light received by the light receiving section 6 becomes.

The light blocking section 15 is a metal plate, and has therefore rigidity even when reduced in thickness, and can surely block the light. Therefore, since the distance between the light emitting section 5 and the light receiving section 6 can be made shorter, it is possible for the biological information measurement device 1 to accurately detect the pulse.

In the side surfaces of the light receiving section 6 facing to the directions crossing the first direction 17, the second side surface 6f, the third side surface 6g, and the fourth side surface 6h not opposed to the light blocking section 15 are opposed to the back lid 3. The passage part 4 is shaped like a plate having a curved surface. A part of the irradiation light 31 undergoes internal reflection inside the passage part 4. The light undergoing the internal reflection inside the passage part 4 is the stray light. A part of the stray light propagates toward the light receiving section 6. The back lid 3 is opaque, and it is possible for the back lid 3 to block the part of the stray light propagating toward the light receiving section 6.

Since the back lid 3 is irradiated with the stray light propagating from the inside of the passage part 4 toward the light receiving section 6 in the Y direction in FIG. 6, the stray light propagating from the inside of the passage part 4 toward the light receiving section 6 in the Y direction fails to reach the light receiving section 6. Since the back lid 3 is also irradiated with the stray light propagating from the inside of the passage part 4 toward the light receiving section 6 in the −Y direction, the stray light propagating from the inside of the passage part 4 toward the light receiving section 6 in the −Y direction also fails to reach the light receiving section 6. Since the back lid 3 is irradiated with the stray light propagating from the inside of the passage part 4 toward the light receiving section 6 in the X direction in FIG. 7, the stray light propagating from the inside of the passage part 4 toward the light receiving section 6 in the X direction fails to reach the light receiving section 6.

As shown in FIG. 6 and FIG. 7, the back lid 3 can also block the reflected light 32 propagating from a place distant from the measurement target part 29 toward the light receiving section 6 besides the above. Since the light receiving section 6 can be prevented from receiving the stray light which turns to the noise component, it is possible for the biological information measurement device 1 to accurately detect the pulse.

As shown in FIG. 6, the passage part 4 is recessed in the inner surface part 4b facing to the light blocking section 15. The side of the light blocking section 15 facing to the passage part 4 protrudes along the inner surface part 4b. It is possible to narrow a gap between the passage part 4 and the light blocking section 15 compared to when, for example, the light blocking section 15 is flat or recessed on the side facing to the passage part 4. Therefore, it is possible to prevent the light receiving section 6 from receiving the stray light which is reflected by the passage part 4 and then passes through the gap between the passage part 4 and the light blocking section 15.

As shown in FIG. 6 and FIG. 7, in the plan view from the first direction 17, the second side surface 6f, the third side surface 6g, and the fourth side surface 6h of the light receiving section 6 and the back lid 3 are separated from each other. Since the gap exists between each of the second side surface 6f, the third side surface 6g, and the fourth side surface 6h of the light receiving section 6 and the back lid 3, it is possible to easily assemble the light receiving section 6 and the back lid 3.

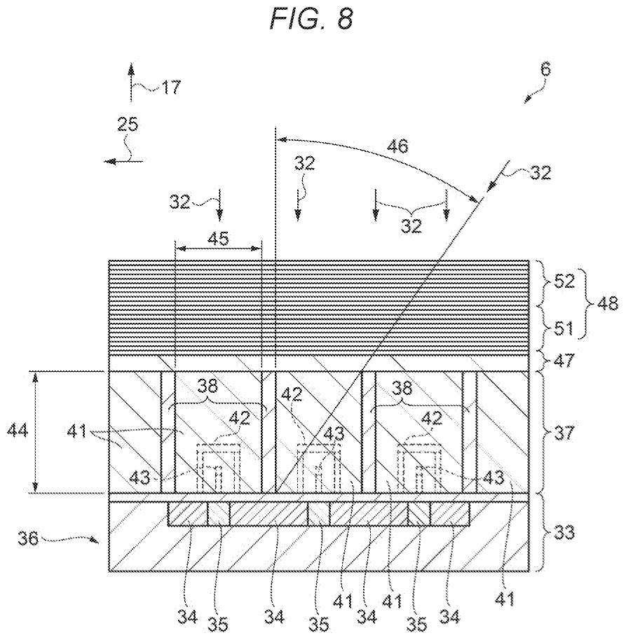

FIG. 8 is a schematic sectional side view showing a structure of the light receiving section. As shown in FIG. 8, the light receiving section 6 is provided with a silicon substrate 33. The silicon substrate 33 is a P-type substrate. Inside the silicon substrate 33, N-type diffusion layers 34 and P-type diffusion layers 35 are alternately arranged in a planar direction on the first direction 17 side. Further, due to the p-n junction between the N-type diffusion layer 34 and the silicon substrate 33, there is formed a photodiode 36. Further, due to the p-n junction between the N-type diffusion layer 34 and the P-type diffusion layer 35, there is formed the photodiode. The N-type diffusion layer 34 forms a cathode of the photodiode, and the P-type diffusion layer 35 and the silicon substrate form an anode.

On the first direction 17 side of the silicon substrate 33, there is disposed an angular limitation filter 37. In the angular limitation filter 37, there are arranged light blocking members 38 at regular intervals in the second direction 25. The light blocking members 38 are each a film thin in the second direction 25. As the material of the light blocking members 38, there is used aluminum, tungsten, or the like. Between the light blocking members 38, there are disposed light transmissive members 41. It is sufficient for the material of the light transmissive member 41 to be able to transmit the reflected light 32 with the wavelength to be received by the photodiode 36. In the present embodiment, for example, silicon dioxide is used as the material of the light transmissive members 41.

In the angular limitation filter 37, there are disposed first interconnections 42 electrically coupled to the N-type diffusion layers 34. Further, there are disposed second interconnections 43 electrically coupled to the P-type diffusion layers 35. In the first interconnections 42 and the second interconnections 43, tungsten is used as a part elongated in the first direction 17. In the first interconnections 42 and the second interconnections 43, aluminum is used as a part elongated in the second direction 25.

Since the intensity of the light of the reflected light 32 which reaches the light blocking member 38 is attenuated, the angle at which the reflected light 32 high in intensity reaches the photodiode 36 is limited within an angle limit 46. The length in the first direction 17 of the light transmissive member 41 is defined as a first length 44. The length in the second direction 25 of the light transmissive member 41 is defined as a second length 45. Further, the angle limit 46 for limiting the reflected light 32 is obtained as arctan((second length 45)/(first length 44)). By setting the first length 44 and the second length 45, the angle limit 46 is set. In the present embodiment, for example, the first length 44 is 5 μm, and the second length 45 is 3 μm. In this case, the angle limit 46 is 31°.

On the first direction 17 side of the angular limitation filter 37, there is disposed a protective film 47. As the material of the protective film 47, there is used silicon dioxide the same as the material of the light transmissive members 41.

On the first direction 17 side of the protective film 47, there is disposed a band-pass filter 48. The band-pass filter 48 is constituted by a long-pass filter 51 formed on the protective film 47 and a short-pass filter 52 formed on the long-pass filter 51. The long pass filter 51 is a filter having a function of passing the light on the long wavelength side, and attenuating the light on the short wavelength side. The short pass filter 52 is a filter having a function of passing the light on the short wavelength side, and attenuating the light on the long wavelength side. In the present embodiment, the band-pass filter 48 passes the light having the wavelength in a range of, for example, 500 nm through 600 nm. The long-pass filter 51 and the short-pass filter 52 are each a thin film filter having thin films stacked on one another. It should be noted that the positions in the first direction 17 of the long-pass filter 51 and the short-pass filter 52 can be reversed from each other.

An outline of a method of manufacturing the light receiving section 6 will be described. Firstly, the photodiode 36 is formed. As the photodiode 36, the N-type diffusion layers 34 and the P-type diffusion layers 35 are formed on the silicon substrate 33 as the P-type substrate. The N-type diffusion layers 34 are each formed by injecting an element of the group V such as phosphorus or arsenic in a predetermined pattern of the silicon substrate 33. The P-type diffusion layers 35 are each formed by injecting an element of the group III such as boron in a predetermined pattern of the silicon substrate 33.

Then, the angular limitation filter 37 is formed. Firstly, a film made of silicon dioxide is deposited using a sputtering process in a step 1. Then, in a step 2, holes are formed using a photolithography process and an etching process. Then, in a step 3, a metal film made of aluminum or tungsten is disposed in the holes and on the film made of silicon dioxide using a sputtering process. Then, in a step 4, the film made of silicon dioxide is planarized using CMP (chemical mechanical polishing).

By repeating the step 1 through the step 4 described above, the light blocking members 38 and the light transmissive members 41 are formed. When forming the interconnections in the planar direction of the silicon substrate 33 in the first interconnections 42 and the second interconnections 43, the metal film formed in the step 3 is formed using the photolithography process and the etching process. Then, the transition to the step 1 is made. In such a manner, the angular limitation filter 37 is formed. The protective film 47 is formed so as to overlap the angular limitation filter 37. As the protective film 47, a film made of silicon dioxide is deposited using a sputtering process.

Then, the band-pass filter 48 is formed so as to overlap the protective film 47. Then, anisotropic dry etching and polishing using the CMP are performed on the protective film 47 to form a tilted surface of a tilted structure. Then, a sputtering process of a titanium oxide film and a sputtering process of a silicon dioxide film are alternately performed to form multilayer thin films on the tilted surface. The titanium oxide film is a thin film high in refractive index, and the silicon dioxide film is a thin film low in refractive index. The film thickness of the titanium oxide films and the film thickness of the silicon dioxide films are adjusted in accordance with the optical characteristics of the long-pass filter 51 and the short-pass filter 52. Due to the processes described hereinabove, the light receiving section 6 is completed.

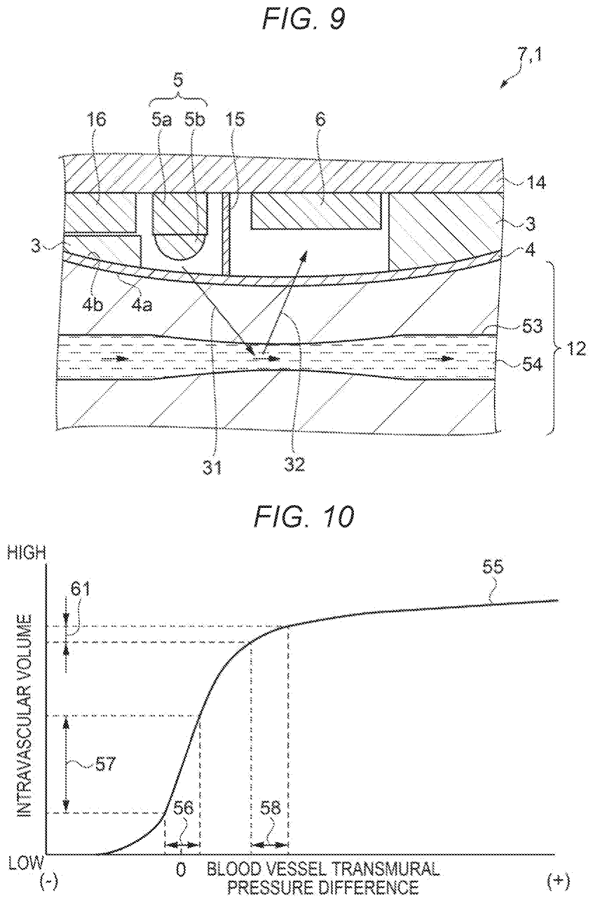

FIG. 9 is a schematic view for explaining a method of detecting the pulsation of the blood vessel. As shown in FIG. 9, inside the arm 12, there is disposed the blood vessel 53 of an arteriole. Inside the blood vessel 53, the blood 54 flows. Due to the pumping of the blood 54, a bulge of the blood vessel 53 propagates. The volume of the blood in the blood vessel 53 of a predetermined length is defined as an intravascular volume. The intravascular volume is proportional to the cross-sectional area of a region where the blood 54 flows in the blood vessel 53. When the blood vessel 53 bulges, the intravascular volume increases, and when the blood vessel 53 deflates, the intravascular volume decreases. The intravascular volume varies in sync with the cardiac motion. Since the cardiac motion coordinates with the pulsation of the blood vessel, the variation in the intravascular volume coordinates with the pulsation of the blood vessel.

A part of the irradiation light 31 emitted from the light emitting section 5 is absorbed by hemoglobin in the blood 54. A part of the irradiation light 31 not absorbed by the hemoglobin is received by the light receiving section 6 as the reflected light 32. When the intravascular volume increases, the proportion of the irradiation light 31 absorbed by the hemoglobin to the irradiation light 31 emitted from the light emitting section 5 increases. When the intravascular volume increases, the reflected light 32 received by the light receiving section decreases. Therefore, the light intensity of the reflected light 32 received by the light receiving section 6 coordinates with the variation in the intravascular volume.

In Masamichi Nogawa et al., Transactions of Japanese Society for Medical and Biological Engineering, volume 49, issue 6, issued by Japanese Society for Medical and Biological Engineering, December 2011, pp. 968-976, there is disclosed information of a relationship between the pressure applied to the blood vessel 53 and the variation in intravascular volume. According to this document, when applying the pressure approximate to the blood pressure to the blood vessel 53, the variation in the intravascular volume increases. FIG. 10 is a diagram for explaining a relationship between a blood vessel transmural pressure difference and the intravascular volume. In FIG. 10, the horizontal axis represents the blood vessel transmural pressure difference. The blood vessel transmural pressure difference is obtained by subtracting “pressure externally applied to the blood vessel” from “average pressure inside the blood vessel.” In the left side of the drawing on the horizontal axis, the pressure externally applied to the blood vessel 53 is made higher compared to the right side of the drawing. When the outer surface part 4a of the passage part 4 is separated from the arm 12, the blood vessel transmural pressure difference becomes in the state in the right side of the drawing on the horizontal axis. When the outer surface part 4a of the passage part 4 depresses the arm 12, the blood vessel transmural pressure difference becomes in the state approximate to “0” on the horizontal axis. In the blood vessel transmural pressure difference, the state of “0” on the horizontal axis means the state in which the average value of the blood pressure inside the blood vessel 53 and the pressure applied by the outer surface part 4a of the passage part 4 to the blood vessel 53 are the same as each other.

The vertical axis represents the intravascular volume, and in the upper side of the drawing, the intravascular volume is higher compared to the lower side of the drawing. A pressure-volume curve 55 represents the relationship between the blood vessel transmural pressure difference and the intravascular volume. The change rate of the pressure-volume curve 55 represents the gradient of the pressure-volume curve 55. When the gradient of the pressure-volume curve 55 is steep, the change rate of the intravascular volume is high, and when the gradient of the pressure-volume curve 55 is gentle, the change rate of the intravascular volume is low. When the blood vessel transmural pressure difference is “0,” the change rate of the intravascular volume is high, and the change rate of the intravascular volume decreases as the blood vessel transmural pressure difference gets away from “0.”

The variation in the blood vessel transmural pressure difference when the contact surface 3a has contact with the arm 12, and the outer surface part 4a of the passage part 4 depresses the arm 12 is defined as a first pressure variation 56. The amplitude of the first pressure variation represents the blood vessel transmural pressure difference varying due to the pumping. The first pressure variation 56 occurs in the vicinity of “0” in the blood vessel transmural pressure difference. Further, the intravascular volume corresponding to the first pressure variation 56 is defined as a first volume variation 57.

The variation in the blood vessel transmural pressure difference when the contact surface 3a is separated from the arm 12 is defined as a second pressure variation 58. The first pressure variation 56 and the second pressure variation 58 are the same in width of the pressure difference variation. In the second pressure variation 58, since the blood vessel 53 is not depressed by the outer surface part 4a of the passage part 4, the second pressure variation 58 is located on the right side in the drawing from the first pressure variation 56. Further, the intravascular volume corresponding to the second pressure variation 58 is defined as a second volume variation 61.

The gradient of the pressure-volume curve 55 in the first pressure variation 56 is steeper than the gradient of the pressure-volume curve 55 in the second pressure variation 58. In other words, the change rate of the pressure-volume curve 55 becomes higher. Therefore, the variation width of the first volume variation 57 becomes larger than the variation width of the second volume variation 61.

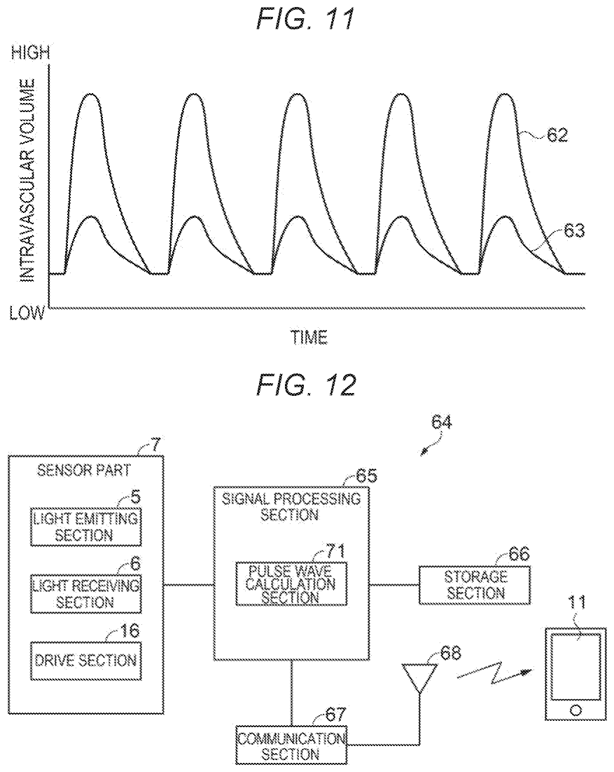

FIG. 11 is a diagram showing the temporal change in the intravascular volume. The horizontal axis in FIG. 11 represents elapse of time, and the time proceeds from the left side in the drawing toward the right side. The vertical axis represents the intravascular volume, and in the upper side of the drawing, the intravascular volume is higher compared to the lower side of the drawing. The first waveform 62 is a waveform corresponding to the first volume variation 57, and the second waveform 63 is a waveform corresponding to the second volume variation 61. The first waveform 62 and the second waveform 63 are similarity shapes. Further, the peak of the intravascular volume is higher in the first waveform 62 than in the second waveform 63. Therefore, by the outer surface part 4a of the passage part 4 depressing the arm 12 to apply the appropriate pressure to the blood vessel 53, the amplitude of the variation of the intravascular volume increases. In this case, it becomes easy for the sensor part 7 to detect the pulsation of the blood vessel 53.

FIG. 12 is a block diagram of electric control of the biological information measurement device. In FIG. 12, the biological information measurement device 1 is provided with a control section 64 for controlling the operation of the biological information measurement device 1. Further, the control section 64 is provided with a signal processing section 65 for performing a variety of types of arithmetic processing, and a storage section 66 for storing a variety of types of information. To the signal processing section 65, there are coupled the sensor part 7 and a communication section 67.

The communication section 67 is provided with a modulation circuit and a demodulation circuit for performing the wireless communication. Further, to the communication section 67, there is coupled an antenna 68. The communication section 67 performs a communication process of Near Field Communication such as Bluetooth (registered trademark) with a terminal device such as a smartphone 11. Specifically, the communication section 67 performs a reception process of a signal from the antenna 68, and a transmission process of a signal to the antenna 68. The function of the communication section 67 can be realized by a processor for communication, or a logic circuit such as an ASIC (Application Specific Integrated Circuit). The communication section 67 wirelessly transmits the pulse information such as the pulse rate calculated by the signal processing section 65 to the smartphone 11 from the antenna 68.

The operator operates the smartphone 11 to perform setup and an instruction of the operation of the biological information measurement device 1. Then, the smartphone 11 transmits the instruction information to the biological information measurement device 1. The communication section 67 receives the instruction information from the smartphone 11. Therefore, the smartphone 11 performs the operation instruction to the biological information measurement device 1, and display of the data such as the pulse wave or the pulse rate detected by the biological information measurement device 1.

The storage section 66 is constituted by a semiconductor memory such as a RAM and a ROM. The storage section 66 stores a program in which a control procedure of the operation of the biological information measurement device 1 and a calculation procedure of the pulse wave are described. Besides the above, the storage section 66 stores the data of the pulse wave signal output by the sensor part 7. In addition, the storage section 66 is provided with a storage area functioning as a work area for the signal processing section 65 to operate or a temporary file, and other variety of storage areas.

The signal processing section 65 is a device for performing a variety of signal processing and a control processing using, for example, the storage area 66 as the work area. The signal processing section 65 is realized by a processor such as a CPU (Central Processing Unit), or a logic circuit such as an ASIC (Application Specific Integrated Circuit).

The signal processing section 65 has a pulse wave calculation section 71. The pulse wave calculation section 71 inputs the data of the pulse wave signal from the sensor part 7 to perform the arithmetic processing of the pulse information. The pulse information is the information such as the pulse rate. Specifically, the pulse wave calculation section 71 performs a frequency analyzing process such as an FFT (fast Fourier transform) on the pulse wave signal to obtain the spectrum of the pulse wave signal. The frequency with the high intensity in the spectrum thus obtained is multiplied by 60 to obtain the pulse rate. It should be noted that the pulse information is not limited to the pulse rate itself, but can also be, for example, the frequency or the period of the pulse wave. Besides the above, it is possible for the pulse information to include data of a temporal change in the pulse rate.

### Second Embodiment

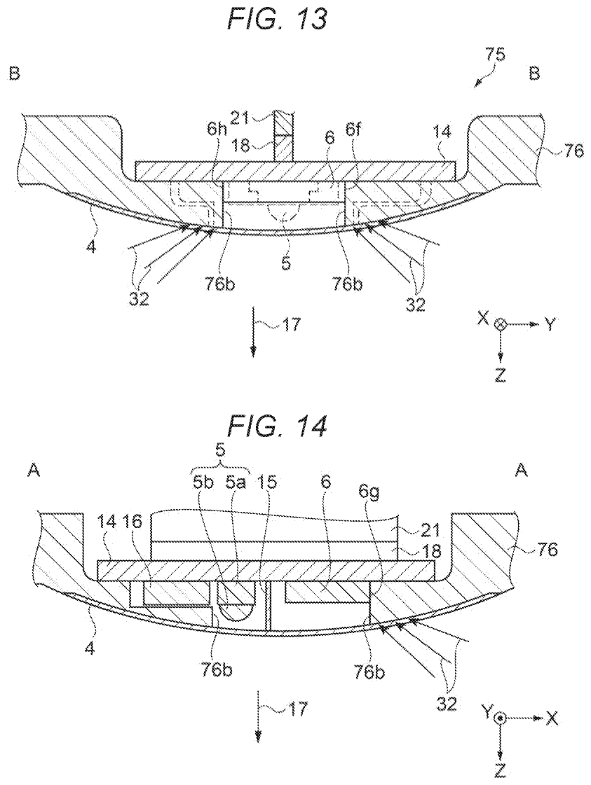

Then, a biological information measurement device according to another embodiment will be described with reference to FIG. 13 and FIG. 14. FIG. 13 and FIG. 14 are each a schematic sectional side view showing a principal part of a configuration of a sensor part and a back lid. FIG. 13 corresponds to a view viewed from a cross-sectional surface side along the line B-B shown in FIG. 3. FIG. 14 corresponds to a view viewed from a cross-sectional surface side along the line A-A shown in FIG. 3. The present embodiment is different from the first embodiment in the point that gaps between the sensor part 7 and the back lid 3 are different. It should be noted that the description of the same point as in the first embodiment will be omitted.

In other words, in the present embodiment, as shown in FIG. 13 and FIG. 14, a biological information measurement device 75 is provided with a back lid 76. The back lid 76 is provided with a hole 76b disposed on the Z direction side of the light emitting section 5 and the light receiving section 6. The hole 76b is blocked by the passage part 4. In the plan view viewed from the first direction 17, the second side surface 6f, the third side surface 6g, and the fourth side surface 6h of the light receiving section 6 have contact with the back lid 76 in the hole 76b.

The shapes of the light receiving section 6 and the back lid 76 are accurately formed to assemble the light receiving section 6 and the back lid 76. The back lid 76 is opaque, and it is possible for the back lid 76 to block the part of the stray light propagating toward the light receiving section 6. Since the back lid 76 is disposed so as to have contact with the light receiving section 6, it is possible for the back lid 76 to prevent the light receiving section 6 from receiving the stray light compared to when the gaps exist between the back lid 76 and the light receiving section 6. In addition, it is also possible for the back lid 76 to block the reflected light 32 propagating from a place distant from the measurement target part 29 toward the light receiving section 6. Since the back lid 76 is disposed so as to have contact with the second side surface 6f, the third side surface 6g, and the fourth side surface 6h of the light receiving section 6, it is possible for the back lid 76 to prevent the light receiving section 6 from receiving the unwanted reflected light 32 compared to when the gaps exist between the back lid 76 and the second side surface 6f, the third side surface 6g, and the fourth side surface 6h of the light receiving section 6.

### Third Embodiment

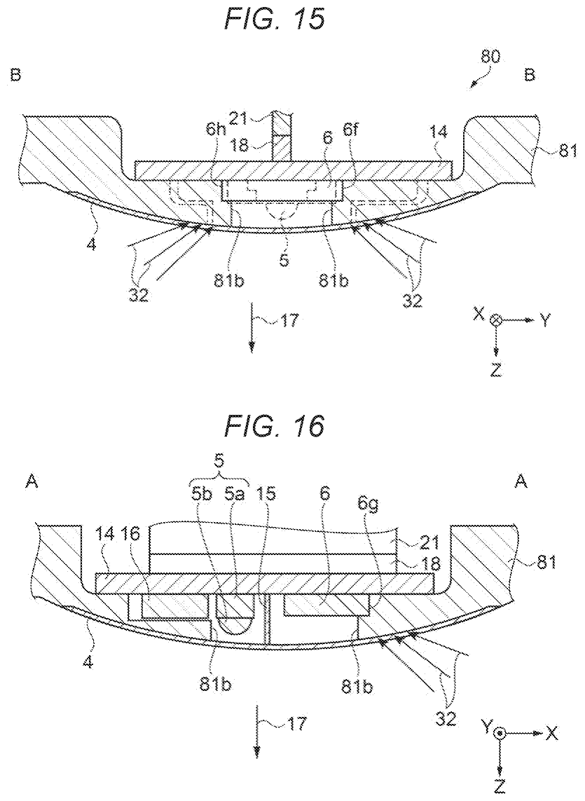

Then, a biological information measurement device according to another embodiment will be described with reference to FIG. 15 and FIG. 16. FIG. 15 and FIG. 16 are each a schematic sectional side view showing a principal part of a configuration of a sensor part and a back lid. FIG. 15 corresponds to a view viewed from a cross-sectional surface side along the line B-B shown in FIG. 3. FIG. 16 corresponds to a view viewed from a cross-sectional surface side along the line A-A shown in FIG. 3. The present embodiment is different from the first embodiment in the point that the light receiving section 6 and the back lid partially overlap each other in the first direction 17 of the light receiving section 6. It should be noted that the description of the same point as in the first embodiment will be omitted.

In other words, in the present embodiment, as shown in FIG. 15 and FIG. 16, a biological information measurement device 80 is provided with a back lid 81. The back lid 81 is provided with a hole 81b disposed on the Z direction side of the light emitting section 5 and the light receiving section 6. The hole 81b is blocked by the passage part 4. In the plan view viewed from the first direction 17, the light receiving section 6 overlaps a part of the back lid 81 on the arm 12 side of the light receiving section 6. The place where the overlap occurs is the outer peripheral side of the light receiving section 6.

Further, the back lid 81 is opaque, and it is possible for the back lid 81 to block the part of the stray light propagating toward the light receiving section 6. On the arm 12 side of the light receiving section 6, a part of the back lid 81 protrudes toward the light receiving section 6 side. The back lid 81 absorbs the stray light with which the protruding part of the back lid 81 is irradiated with. Therefore, it is possible for the back lid 81 to prevent the light receiving section 6 from receiving the stray light. In addition, a part of the reflected light 32 propagating obliquely to the first direction 17 is blocked by the back lid 81. In addition, it is possible for the back lid 81 to prevent the light emitting section 6 from receiving the outside light emitted by the sun, a fluorescent light, or the like. It is also possible for the back lid 81 to block the reflected light 32 propagating from a place distant from the measurement target part 29 toward the light receiving section 6. Therefore, it is possible for the back lid 81 to prevent the light receiving section 6 from receiving the unwanted reflected light 32.

### Fourth Embodiment

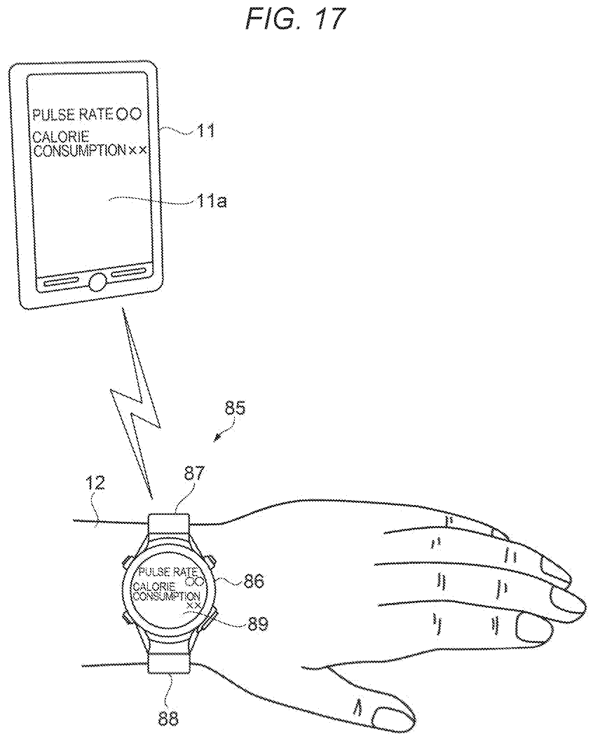

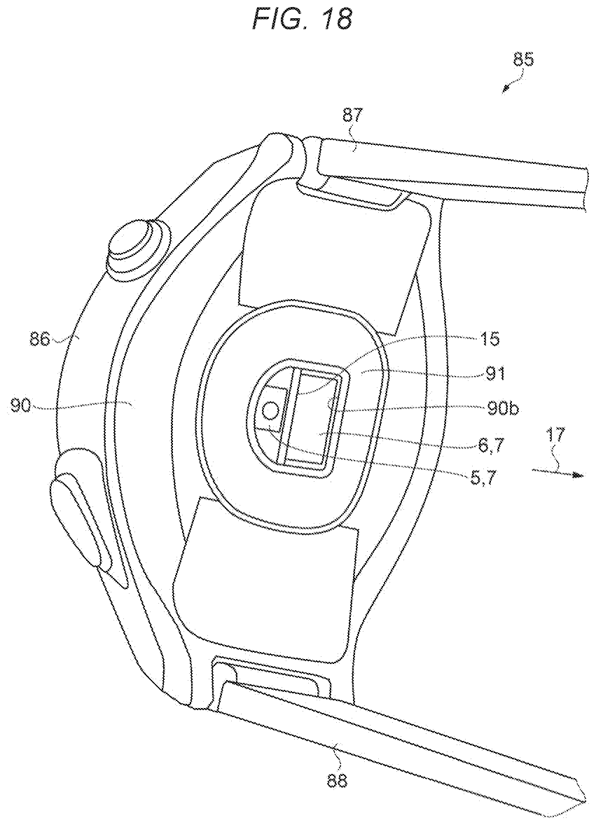

Then, a biological information measurement device according to another embodiment will be described with reference to FIG. 17 and FIG. 18. FIG. 17 is a schematic view for explaining a mounting state of the biological information measurement device. FIG. 18 is a schematic perspective view showing a configuration of the biological information measurement device. The present embodiment is different from the first embodiment in the point that the biological information measurement device is provided with a display section. It should be noted that the description of the same point as in the first embodiment will be omitted.

As shown in FIG. 17, the appearance of the biological information measurement device 85 is similar to an appearance of a watch. The biological information measurement device 85 is mounted on the arm 12 of the user and detects the biological information such as the pulse wave information. The biological information measurement device 85 has a case 86, a first band 87, and a second band 88. The first band 87, and the second band 88 are for mounting the case 86 on the user. It should be noted that the description will be presented citing when the biological information measurement device 85 is a watch type pulse monitor to be mounted on the arm 12 as an example. This example is not a limitation. For example, it is also possible for the biological information measurement device 85 to be a device mounted on a finger, an upper arm, a chest, or the like to detect the biological information. Further, the biological information to be the detection target of the biological information measurement device 85 is not limited to the pulse wave. For example, the biological information measurement device 85 can also be a device for detecting the oxygen saturation in the blood, the body temperature, the heart rate, and the blood pressure besides the pulse wave.

The case 86 is provided with a first display section 89 such as an LCD (Liquid Crystal Display). On the first display section 89, there are displayed a variety of types of information such as the pulse rate or the calorie consumption. The biological information measurement device 85 is coupled to the smartphone with the communication to perform data transactions. The smartphone 11 is provided with a second display section 11a such as an LCD. On the second display section 11a of the smartphone 11, there are displayed a variety of types of information such as the pulse rate and the calorie consumption. It should be noted that the arithmetic processing of the information such as the pulse rate and the calorie consumption can be executed by the biological information measurement device 85, or at least a part of the arithmetic processing can be executed by the smartphone 11.

As shown in FIG. 18, on the opposite side of the case 86 to the first display section 89, there is disposed a back lid 90. A passage part 91 through which the light can pass is disposed at the center of the back lid 90. Inside the case 86, there are disposed the sensor part 7 provided with the light emitting section 5, and the light receiving section 6, and the light blocking section 15, and so on. The back lid 90 is provided with a hole 90b disposed in a place opposed to the light emitting section 5 and the light receiving section 6. The hole 90b is blocked by the passage part 91. The passage part 91 is transparent, and therefore, the light emitting section 5, the light receiving section 6, and a light blocking section 15 can be seen through the hole 90b. Therefore, in FIG. 18, the light emitting section 5, the light receiving section 6, and the light blocking section 15 are represented by the solid lines.

In the plan view from the first direction 17, the light blocking section 15 is a metal plate disposed between the light emitting section 5 and the light receiving section 6. The side surfaces not opposed to the light blocking section 15 in the side surfaces facing to the direction crossing the first direction 17 out of the light receiving section 6 are opposed to the back lid 90.

In the biological information measurement device 85, the light blocking section 15 is a metal plate, and has therefore rigidity even when reduced in thickness, and can surely block the light. Therefore, since the distance between the light emitting section 5 and the light receiving section 6 can be made shorter, it is possible for the biological information measurement device 85 to accurately detect the pulse. It is possible for the back lid 90 to block a part of the stray light propagating toward the light receiving section 6. In addition, it is also possible for the back lid 90 to block the reflected light 32 propagating from a place distant from the measurement target part 29 toward the light receiving section 6.

It should be noted that the present embodiment is not limited to the embodiment described above, but a variety of modifications or improvements can also be added by those skilled in the art within the technical concept of the present disclosure. Some modified examples will be described below.

### Modified Example 1

In the first embodiment described above, in the light blocking section 15, the side facing to the passage part 4 protrudes. When the inner surface part 4b on the side facing to the light blocking section 15 of the passage part 4 is a plane, the side facing to the passage part 4 of the light blocking part 15 can be a plane. When the inner surface part 4b on the side facing to the light blocking section 15 of the passage part 4 protrudes, the side facing to the passage part 4 of the light blocking part 15 can be recessed. The shape of the light blocking section 15 can be made to have a shape corresponding to the shape of the passage part 4.

### Modified Example 2

In the first embodiment described above, the biological information measurement device 1 is mounted on the arm 12 of the human body. The biological information measurement device 1 can also be mounted on other regions than the arm 12. For example, it is also possible for the biological information measurement device 85 to be a device mounted on a finger, an upper arm, a chest, or the like to detect the biological information. It is also possible for the biological information measurement device 1 to be mounted on an animal other than the human. Further, the biological information to be the detection target of the biological information measurement device 1 is not limited to the pulse wave. For example, the biological information measurement device 1 can also be a device for detecting the oxygen saturation in the blood, the body temperature, the heart rate, and the blood pressure besides the pulse wave.

The contents derived from the embodiments will hereinafter be described.

The biological information measurement device includes a light emitting section configured to emit irradiation light with which a living body is irradiated, a light receiving section configured to receive reflected light which is the irradiation light reflected by the living body, a passage part which the irradiation light and the reflected light pass through, a light blocking section configured to block the irradiation light propagating from the light emitting section toward the light receiving section, and a back lid which is opaque, and supports the passage part, wherein the light blocking section is a metal plate disposed between the light emitting section and the light receiving section in a plan view viewed from a first direction from the light emitting section toward the passage part, and a side surface which is one of side surfaces of the light receiving section, which faces to a direction crossing the first direction, and which fails to be opposed to the light blocking section, is opposed to the back lid.

According to this configuration, the biological information measurement device is provided with the light emitting section and the light receiving section. The light emitting section emits the irradiation light toward the living body. The passage part is disposed between the light emitting section and the living body. The irradiation light propagates toward the living body passing through the passage part. The irradiation light propagating toward the living body is reflected by the living body. A part of the reflected light reflected by the living body propagates toward the light receiving section. The passage part is disposed between the light emitting section and the living body. The reflected light propagates toward the light receiving section passing through the passage part. The light receiving section receives the reflected light.

In the blood vessel of the living body, the blood absorbs a part of the irradiation light. In the blood vessel, since the blood flows as a pulsating flow, the reflected light has a temporal change in intensity reflecting the pulsating flow of the blood. The biological information measurement device measures the reflected light to detect the pulsation of the blood vessel. Since the irradiation light does not have the temporal change in intensity reflecting the pulse, the irradiation light received by the light receiving section turns to the noise component. When the light receiving section does not receive the irradiation light, the accuracy of detecting the pulse becomes higher.

A part of the irradiation light propagates toward the light receiving section. The light blocking section is disposed between the light emitting section and the light receiving section. The light blocking section blocks the irradiation light propagating toward the light receiving section. The light blocking section prevents the irradiation light from being received by the light receiving section. A part of the irradiation light undergoes internal reflections inside the passage part. The light undergoing the internal reflection inside the passage part is referred to as the stray light. A part of the stray light propagates toward the light receiving section. The side surface of the light receiving section not opposed to the light blocking section is opposed to the back lid. Further, the back lid is opaque, and it is possible for the back lid to block the part of the stray light propagating toward the light receiving section. Since the light receiving section can be prevented from receiving the stray light which turns to the noise component, it is possible for the biological information measurement device to accurately detect the pulse.

The accuracy of detecting the pulse is higher when the intensity of the reflected light to be received by the light receiving section is higher compared to when the intensity of the reflected light is lower. Therefore, the shorter the distance between the light emitting section and the living body is, the higher the intensity of the irradiation light with which the living body is irradiated becomes. The shorter the distance between the light emitting section and the living body is, the higher the intensity of the reflected light received becomes.

In a triangle having the light emitting section, the living body, and the light receiving section as vertexes, the distance between the light emitting section and the living body corresponds to the propagation distance of the irradiation light. The distance between the light receiving section and the living body corresponds to the propagation distance of the reflected light. When the distance between the light emitting section and the light receiving section is short, the total distance of the propagation distance of the irradiation light and the propagation distance of the reflected light can be shortened compared to when the distance between the light emitting section and the light receiving section is long. Since the irradiation light and the reflected light do not have a converging property, the shorter the propagation distances of the irradiation light and the reflected light are, the higher the intensity of the reflected light received by the light receiving section becomes.

The light blocking section is a metal plate, and has therefore rigidity even when reduced in thickness, and can surely block the light. Therefore, since the distance between the light emitting section and the light receiving section can be made shorter, it is possible for the biological information measurement device to accurately detect the pulse.

In the biological information measurement device described above, in a plan view viewed from the first direction, the side surface of the light receiving section and the back lid may be separated from each other.

According to this configuration, the side surface of the light receiving section and the back lid are separated from each other. In other words, since a gap exists between the side surface of the light receiving section and the back lid, it is possible to easily assemble the light receiving section and the back lid.

In the biological information measurement device described above, in a plan view viewed from the first direction, the side surface of the light receiving section and the back lid may have contact with each other.

According to this configuration, the side surface of the light receiving section and the back lid have contact with each other. When the shapes of the light receiving section and the back lid can accurately be formed, the light receiving section and the back lid can be assembled. Since the back lid is disposed at a place close to the light receiving section, it is possible for the back lid to prevent the light receiving section from receiving the stray light.

In the biological information measurement device described above, in a plan view viewed from the first direction, the light receiving section and a part of the back lid may overlap each other on the living body side of the light receiving section.

According to this configuration, the part of the back lid protrudes toward the light receiving section on the living body side of the light receiving section. The stray light with which the part of the back lid is irradiated is absorbed by the back lid. Therefore, it is possible for the back lid to prevent the light receiving section from receiving the stray light.

In the biological information measurement device described above, the passage part may be recessed in an inner surface facing to the light blocking section, and a side of the light blocking section facing to the passage part may protrude along the inner surface.

According to this configuration, the light blocking section is disposed between the light emitting section and the light receiving section. The passage part is disposed on the living body side of the light emitting section and the light receiving section. Therefore, the passage part is disposed on the living body side of the light blocking section. The passage part is recessed in an inner surface facing to the light blocking section. A side of the light blocking section facing to the passage part protrudes along the inner surface. In this case, it is possible to narrow a gap between the passage part and the light blocking section compared to when the light blocking section is flat or recessed on the side facing to the passage part. Therefore, it is possible to prevent the light receiving section from receiving the stray light which is reflected by the passage part and then passes through the gap between the passage part and the light blocking section.

What is claimed is:

1. A biological information measurement device comprising:
a light emitting section configured to emit irradiation light with which a living body is irradiated;
a light receiving section configured to receive reflected light which is the irradiation light reflected by the living body;
a passage part which the irradiation light and the reflected light pass through;
a light blocking section configured to block the irradiation light propagating from the light emitting section toward the light receiving section; and
a back lid which is opaque, and supports the passage part, wherein
the light blocking section is a metal plate disposed between the light emitting section and the light receiving section in a plan view viewed from a first direction from the light emitting section toward the passage part, and
a side surface which is one of side surfaces of the light receiving section, which faces to a direction crossing the first direction, and which fails to be opposed to the light blocking section, is opposed to the back lid.

2. The biological information measurement device according to claim 1, wherein
in a plan view viewed from the first direction,
the side surface of the light receiving section and the back lid are separated from each other.

3. The biological information measurement device according to claim 1, wherein
in a plan view viewed from the first direction,
the side surface of the light receiving section and the back lid have contact with each other.

4. The biological information measurement device according to claim 1, wherein
in a plan view viewed from the first direction,
the light receiving section and a part of the back lid overlap each other on the living body side of the light receiving section.

5. The biological information measurement device according to claim 1, wherein
the passage part is recessed in an inner surface facing to the light blocking section, and
a side of the light blocking section facing to the passage part protrudes along the inner surface.
